(19) United States
(12) Patent Application Publication
(10) Pub. No.: US 2024/0358871 A1
(43) Pub. Date: Oct. 31, 2024

(54) DEVELOPMENT OF AN AEROSOL-SURFACE INTERFACE SANITIZATION SYSTEM FOR SURFACE SANITIZATION

(71) Applicant: Nano and Advanced Materials Institute Limited, Hong Kong (HK)

(72) Inventors: Tsz Wai NG, Hong Kong (HK); Wah Kit CHEUK, Hong Kong (HK); Sum Yi MA, Hong Kong (HK); Chun Hay KO, Hong Kong (HK); Cheuk Ka POON, Hong Kong (HK)

(21) Appl. No.: 18/637,452

(22) Filed: Apr. 17, 2024

Related U.S. Application Data
(60) Provisional application No. 63/497,431, filed on Apr. 20, 2023.

(51) Int. Cl. A61L 2/22; A61L 9/14; A61L 101/48

(52) U.S. Cl. CPC A61L 2/22; A61L 9/14; A61L 2101/48

(57) ABSTRACT

An aerosol-surface interface sanitization system comprising a non-toxic, non-corrosive and biocompatible sanitization formulation, and a nebulizer selected from jet-nebulizer, ultrasonic nebulizer or spray gun. This system is designed for interior environment application, and is applicable for both airborne and surface disinfection. The sanitization formulation is formed by membrane emulsification technique to optimize nebulization efficiency through adjustment in viscosity, droplet size, dispersion area and settlement rate, while having a high antiviral, antifungal and antibacterial activity.

### CROSS-REFERENCE TO RELATED APPLICATION

The present application claims priority from a U.S. provisional patent application Ser. No. 63/497,431 filed Apr. 20, 2023, and the disclosures of which are incorporated by reference in their entirety.

### TECHNICAL FIELD

The present invention relates to an automated airborne sanitization system comprising a specifically formulated sanitizer and a nebulizer. More specifically, the sanitizer formulation is non-toxic, non-corrosive and biocompatible, and the system is optimized for indoor environment application.

### BACKGROUND OF THE INVENTION

The emergence and outbreak of infectious diseases such as SARS and COVID-19 has highlighted the need for effective sanitization systems to minimize the threat of spread of diseases through airborne and surface pathogens.

However, the disinfection efficiency of commercial products available in the market is usually unsatisfactory as the particle size of the aerosols is not optimized for a complete disinfection of every corner of the vehicle cabin. In addition, these commercial products are generally not formulated to be effective against mold/fungi, bacteria and virus at the same time.

While sanitizers generally kill harmful surface microbes, the efficiency is affected by the composition of form of the sanitizer composition, and its spraying method. Also, most of the commercial sanitization products may not consider the aerodynamic properties of vaporized sanitizer droplets. Therefore, efficiency in indoor environment disinfection may be limited.

In addition, traditional sanitization methods such as manual wiping or spraying could be time-consuming and labor-intensive, and may not be efficient in reaching all surfaces and corners of interior environments. As such, the development of an automated sanitization system is also crucial.

### SUMMARY OF THE INVENTION

The present invention provides a surface-aerosol interface sanitization (ASIS) system for automated sanitization of interior environments, which comprises a nebulizer selected from a jet or ultrasonic nebulizer configured to emit droplets with sizes of 1 μm to 14 μm or a spray gun configured to emit droplets of 9 μm to 80 μm, and a non-toxic, non-corrosive and biocompatible sanitizer formulation comprising an organic acid, a peptide with antiviral and antimicrobial properties, a polymer binder, a surfactant and an essential oil. The sanitization formulation is an emulsion formed by membrane emulsification through a membrane emulsifier, where the emulsified particles have a particle size of 0.05 μm to 5 μm, configured for nebulization by the jet or ultrasonic nebulizer or the spray gun. The sanitization formulation has a viscosity of 15 mPas to 25 mPas such that the nebulized droplets are configured to be dispersed over an area of approximately 1 cubic meter without evaporating, and have a settlement rate of 0.3 cm/s to 3 cm/s. The sanitization formulation further has at least 99% antibacterial activity against Enterococcus hirae, Escherichia coli, Pseudomonas aeruginosa, Staphylococcus aureus and Enterococcus gaecium; at least 95% antifungal activity against Aspergillus niger, Candida albicans and Aspergillus brasiliensis; and at least 99% antiviral activity against Influenza A virus H1N1.

In one embodiment, the organic acid in the sanitizer formulation is selected from acetic acid, citric acid, malic acid, tartaric acid, lactic acid, succinic acid or oleic acid.

In another embodiment, the peptide with antiviral and antimicrobial properties in the sanitizer formulation is selected from nisin, poly-L-lysine, lysozyme, lysostaphin, microcin, colicin or enterocin.

In other embodiment, the polymer binder in the sanitizer formulation is selected from chitosan, zein, gelatin, cellulose, pectin, alginate, acrylic latex, polyurethane or cyclodextrin.

In yet another embodiment, the surfactant in the sanitizer formulation is selected from polysorbate-based surfactants; polyglyceryl ester-based surfactants; or polyglucose.

In another embodiment, the essential oil in the sanitizer formulation is selected from thyme oil, tea tree oil, cedar wood oil, ginger oil or lemon oil.

In other embodiment, the surface contact angle of the nebulized formulation droplets is less than 32%; and wherein the duration of settlement of the nebulized formulation on a surface is at least 2 hours.

### BRIEF DESCRIPTION OF DRAWINGS

FIG. 1 is a schematic diagram showing the process of membrane emulsification.

FIG. 2 shows the chemical structures of selected major components in the sanitizer formulation, including acetic acid, nisin, thymol and chitosan.

FIG. 3 shows an example showing the preparation of formulation AF37 with an overhead stirrer.

FIG. 4 shows an Shirasu-Porous-Glass membrane emulsifier and its compartments.

FIG. 5 shows the procedures of standard test EN 1276 for testing the antibacterial activity of a formulation against Staphylococcus aureus.

FIG. 6 shows the spread plate method using tryptic soy agar. The photo on the left shows the tryptic soy agar plate before overnight incubation; and the photo on the right shows the tryptic soy agar plate with Staphylococcus aureus colonies after overnight incubation.

FIG. 7 show the procedures of standard test EN 1650 for testing the antifungal activity of a formulation against Aspergillus niger.

FIG. 8 shows the Owgel WH-702 jet-nebulizer and K5-Pro Spray Gun.

FIG. 9 shows the setup of Spraytec laser diffraction system (left), and the example of data of spray droplet size distribution obtained (right).

FIG. 10 shows 2×2 cm² leather used as carrier for testing surface inactivation efficiency of the formulations.

FIG. 11 is a schematic diagram of procedures of the evaluation of surface inactivation efficiency.

FIG. 12 shows the setup for testing the in-house surface inactivation efficiency of a vaporized formulation.

FIG. 13 shows the procedures modified from Technical Standard for Disinfection (2002) Section 2.2.3.

FIG. 14 shows the sampler pump and BioSampler used in air sample collection.

FIG. 15 shows a Multisizer 4e Coulter Counter.

### DETAILED DESCRIPTION

To optimize the efficiency of both airborne and surface sanitization through automated application and inactivation of wide range of microbes and viruses, while maintaining biocompatibility and minimizing corrosion and toxicity, the current surface-aerosol interface sanitization system is developed, comprising a specifically formulated sanitizer and a nebulizer which can be selected from jet-nebulizer, ultrasonic nebulizer or spray gun.

The sanitizer reformulation comprises an organic acid, a biopolymer, a surfactant, an Essential oil and a peptide with antimicrobial and antiviral properties, and is an emulsion formed by membrane emulsification through a membrane emulsifier, and the emulsified particles have a particle size of 0.05 μm to 5 μm configured to be nebulized by the jet-nebulizer, ultrasonic nebulizer or the spray gun to minimize the disruption of the dispersed phase by the nebulizer; wherein the sanitizer formulation has a viscosity of 15 mPas to 25 mPas such that the droplets are configured to be dispersed over an area of approximately 1 cubic meter without evaporating and having a settlement rate of 0.3 cm/s to 3 cm/s.

The sanitizer formulation has at least 99% antibacterial activity against Enterococcus hirae, Escherichia coli, Pseudomonas aeruginosa, Staphylococcus aureus and Enterococcus gaecium; at least 95% antifungal activity against Aspergillus niger, Candida albicans and Aspergillus brasiliensis; and at least 99% antiviral activity against Influenza A virus H1N1.

The organic acid in the sanitizer formulation is selected from acetic acid, citric acid, malic acid, tartaric acid, lactic acid, succinic acid or oleic acid.

The peptide with antiviral and antimicrobial properties in the sanitizer formulation is selected from nisin, poly-L-lysine, lysozyme, lysostaphin, microcin, colicin or enterocin.

The polymer binder in the sanitizer formulation is selected from chitosan, zein, gelatin, cellulose, pectin, alginate, acrylic latex, polyurethane or cyclodextrin.

The surfactant in the sanitizer formulation is selected from polysorbate-based surfactants including Tween®80, Tween®60, Tween®20, Span®80, Span®60 or Span®20; polyglyceryl ester-based surfactants including TEGO®solve 61; or polyglucose.

The essential oil in the sanitizer formulation is selected from thyme oil, tea tree oil, cedar wood oil, ginger oil or lemon oil.

The surface contact angle of the nebulized formulation droplets is less than 32%; and wherein the duration of settlement of the nebulized formulation on a surface is at least 2 hours, which maximizes the staying effect of the sanitizer formulation on any surface.

Natural microbial peptide, such as nisin, is effective in microbial inactivation under acidic condition by inducing pores formation on the bacterial cell membrane which leads to loss of cellular ions.

Essential oil disinfects fungi by penetrating into the lipids of cell and mitochondrial membrane and causing leakages of essential components in fungal cell.

Surfactants are specifically chosen to highly solubilize essential oils while producing little foam during the process.

Biopolymer binder is included to improve the adhesion of the nebulized formulation, and hence increasing the contact between the nebulized formulation to the target surface.

Organic acid aids in microbial inactivation by inducing protein unfolding and DNA damage. The low pH of organic acid also favors the stability of natural microbial peptide.

All the selected ingredients are major natural products with low inhalation and dermal toxicity for the sake of safety during application, as proven by the compliance of the vapor formulation with acute inhalation toxicity standard, RoHS, SVHC, harmful VOC, skin irritation and acute dermal toxicity standards shown below. In addition, as is shown in the test results below, the sanitizer is compatible with metals in indoor environment as it causes no corrosion on stainless steel and only slight corrosion on aluminum.

Aerosol size of the vaporized sanitizer also plays an important role in the sanitization of surface microbes as it affects the aerodynamic properties. If the aerosol size is too small, the vaporized sanitizer mainly follows the airflow movement and may not contact the target surface. While if the aerosol size is too large, the settling velocity of the vapor will be too fast and the vapor settle down before reaching the target surface.

Viscosity is the main factor controlling the aerosol size. The aerosol size of the vaporized sanitizer is controlled in a defined range by adjusting the viscosity of formulation and thus can be easily distributed by airflow in indoor environment. The currently claimed ASIS technology precisely tunes the viscosity of the sanitizer so that the sanitizer can be nebulized by a commercially available nebulizer or spray gun.

The addition of polymer binder in the sanitizer favors the adhesion of the aerosol onto the surface, maximizing the sanitization efficiency.

Membrane emulsification technology is used to prepare the formulations because it allows incorporation of different types of natural bactericidal and fungicidal materials, regardless of their hydrophilicity or hydrophobicity, in a simple and fast operation.

The emulsion is produced by forcing the formulation passing through membrane pore under pressure. The droplet size of the sanitizer can be easily controlled with the use of membrane with different pore sizes.

### EXAMPLES

### Example 1: Membrane Emulsification

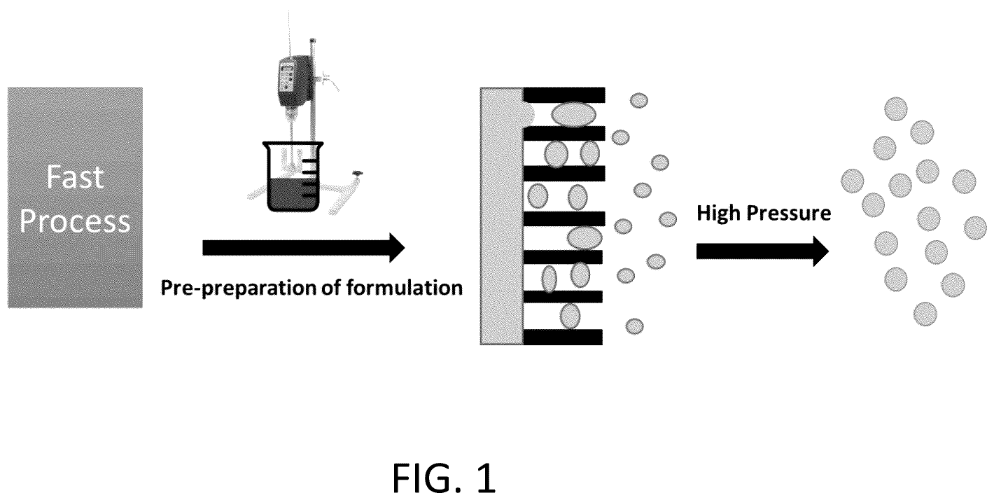

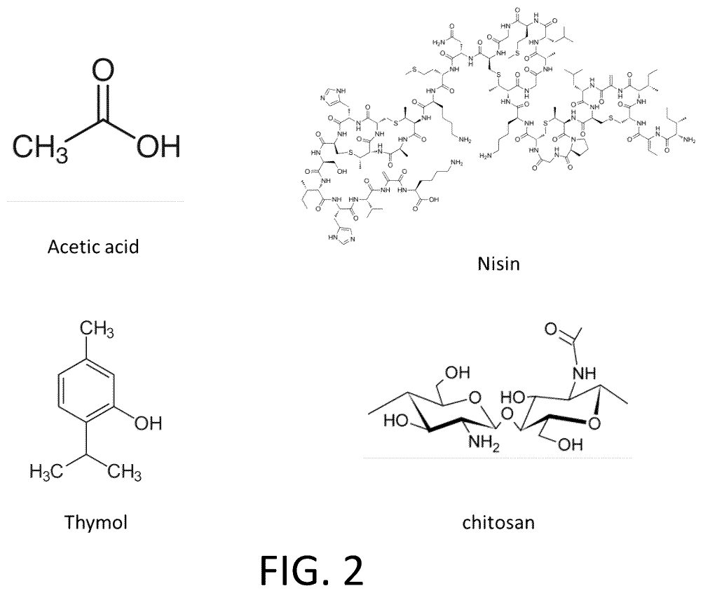

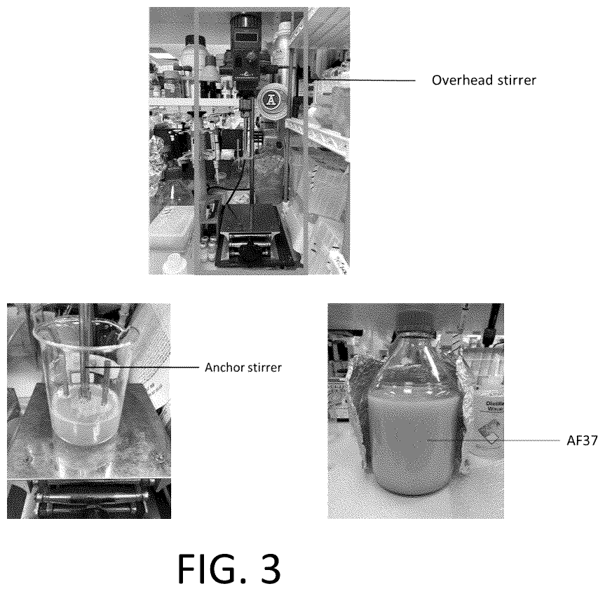

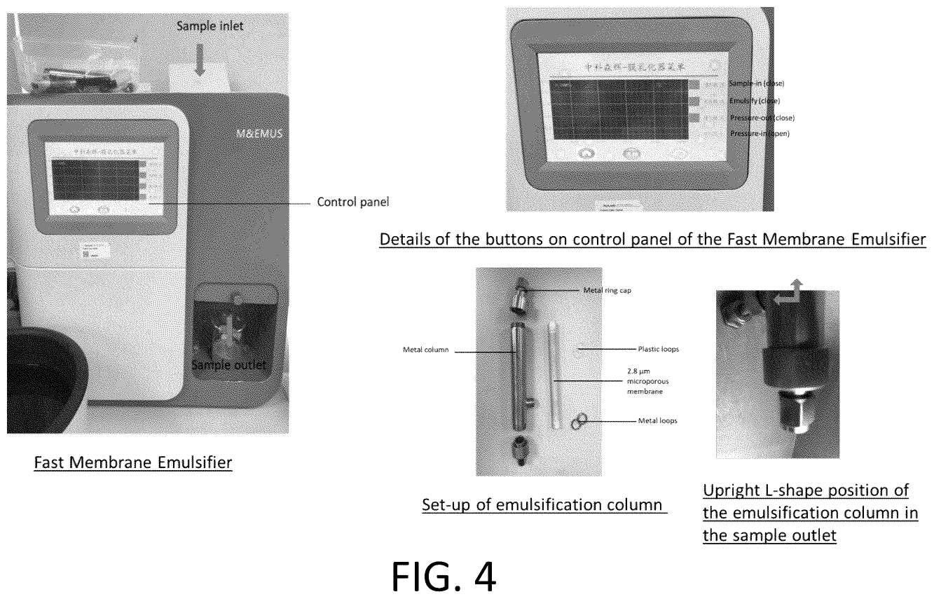

A stable emulsion was produced with simple and fast operation process with the use of membrane emulsification which forced the formulation to pass through a membrane under high pressure (FIG. 1). To produce the formulation, biopolymer, organic acid, surfactant and antimicrobial peptide (FIG. 2) were added into water and mixed well with an overhead stirrer in a defined ratio (FIG. 3). Then the essential oil was added into the solution dropwise and mixed for 10 min. Afterward, the solution was emulsified by a Shirasu-porous-glass (SPG) membrane emulsifier with a membrane of pore size 2.8 μm under pressure of 0.4 MPa and each solution was emulsified for 3 times (FIG. 4). The percentage of each component in each formulation was shown in Table 1 below.

TABLE 1

| | Organic | Antimicrobial | Essential | | |
|---|---|---|---|---|---|
| Formulation | acid (%) | peptide (%) | oil (%) | Biopolymer (%) | Surfactant (%) |
| AF37 | 5 (Acetic | 1 (Nisin | 1 (Thyme) | 0.2 (Chitosan) | 5 (TEGO |
| | acid) | E234) | | | Solve 61) |
| AF38 | 5 (Acetic | 1 (Nisin | 2 (Cedar | 0.2 (Chitosan) | 10 (TEGO |
| | acid) | E234) | wood) | | Solve 61) |
| AF39 | 5 (Acetic | 1 (Nisin | 2 (Tea Tree) | 0.2 (Chitosan) | 10 (TEGO |
| | acid) | E234) | | | Solve 61) |
| AF40 | 5 (Acetic | 1 (Nisin | 1 (Thyme) + | 0.2 (Chitosan) | 10 (TEGO |
| | acid) | E234) | 1 (Tea Tree) | | Solve 61) |
| AF41 | 5 (Acetic | 1 (Nisin | 1 (Tea Tree) + | 0.2 (Chitosan) | 10 (TEGO |
| | acid) | E234) | 1 (Cedar | | Solve 61) |
| | | | wood) | | |
| AF42 | 5 (Acetic | 1 (Nisin | 1 (Thyme) + | 0.2 (Chitosan) | 10 (TEGO |
| | acid) | E234) | 1 (Cedar | | Solve 61) |
| | | | wood) | | |
| AF43 | 1 (Acetic | 0.4 (Nisin | 1 (Thyme) + | 0.15 (Chitosan) | 6.8 (TEGO |
| | acid) | E234) | 1 (Cedar | | Solve 61) |
| | | | wood) | | |
| ASIS002 | 1 (Acetic | 0.4 (Nisin | 1 (Thyme) | 0.15 (Chitosan) | 2 (TEGO |
| | acid) | E234) | | | Solve 61) |
| Sanimood001 | 1 (Citric | 3 (Nisin | — | 0.1 (Chitosan) | 2 (Polyglucose) |
| | acid) | E234) | | | |
| NP15 | 1.5 (Citric | 2 (Nisin | — | 2 (2- | — |
| | acid) | E234) | | Hydroxypropyl- | |
| | | | | β-cyclodextrin) | |

### Example 2: Testing Antibacterial Activity Against S. aureus

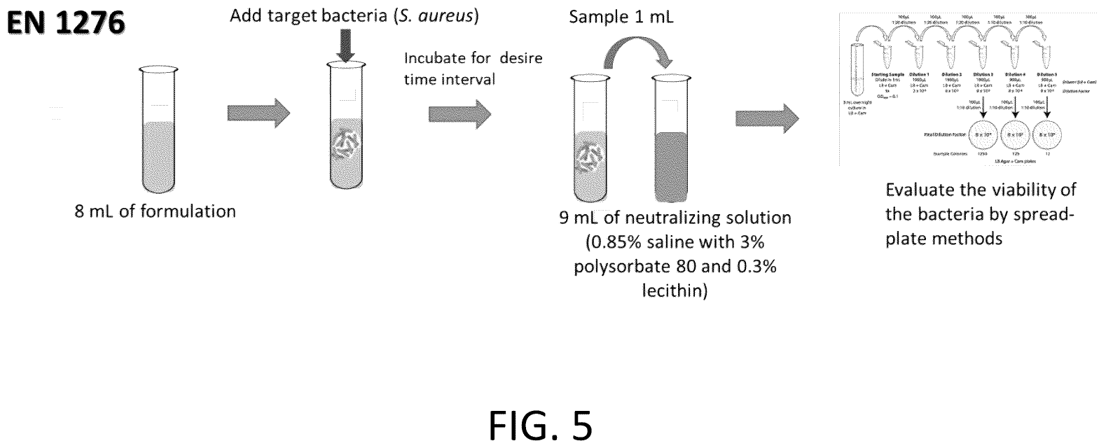

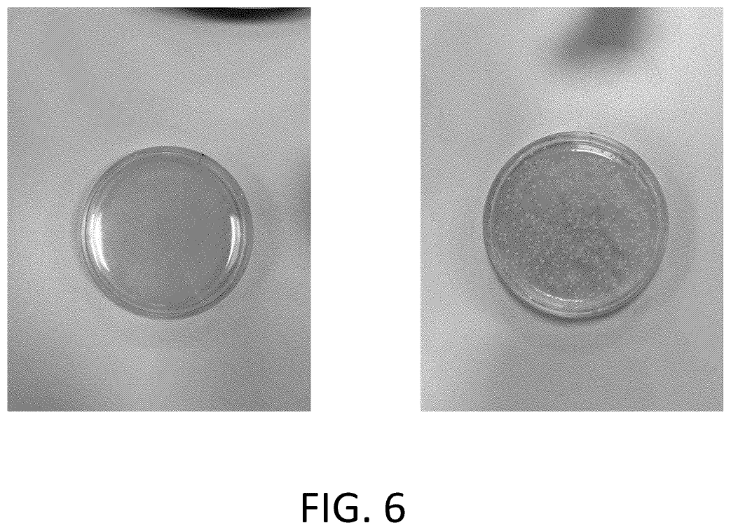

The antibacterial activity of the formulation was evaluated according to the standard test EN 1276 (FIG. 5). In brief, 2 mL of Staphylococcus aureus (S. aureus) was added into 8 mL of formulation. One mL of the formulation was sampled immediately and added into 9 mL of neutralizing solution. After 5 min, another 1 mL of the formulation was sampled again and added into 9 mL of neutralizing solution. The neutralizing solution was made up of 0.85% saline with 3% polysorbate 80 and 0.3% lecithin. Finally, the viability S. aureus of the neutralized sample was determined with the spread plate method using tryptic soy agar (FIG. 6) as culture medium. The antibacterial activity of the formulations was listed in Table 2.

TABLE 2
(Antibacterial activity of different formulation against S. aureus):

| Formulation | Antibacterial activity (%) |
|---|---|
| AF37 | >99.9 |
| AF38 | >99.9 |
| AF39 | >99.9 |
| AF40 | >99.9 |
| AF41 | >99.9 |
| AF42 | >99.9 |
| AF43 | >99.9 |
| ASIS 002 | >99.9 |

### Example 3: Testing Antibacterial Activity Against Different Bacteria

The antibacterial effect of AF42 was tested in accordance with international standard EN 1276: 2019 Chemical disinfectants and antiseptics—Quantitative suspension test for the evaluation of bactericidal activity of chemical disinfectants and antiseptics used in food, industrial, domestic and institutional areas.

The results are shown in Table 3 below.

TABLE 3
(Results of antibacterial activity of
AF42 against different bacteria):

| | | Counts | |
|---|---|---|---|
| | | of test | Percentage |
| | Counts | sample at | reduction |
| | of test | concentration | (log |
| Test microorganism | suspension | 80% | reduction) |
| Enterococcus hirae | 158,500,000 | 750 | >99.99% |
| (ATCC 10541) | Log 8.20 | Log 2.88 | (5.32) |
| Escherichia coli | 152,000,000 | <10 | >99.99% |
| (ATCC 10536) | Log 8.18 | <Log 1.00 | (>7.18) |
| Pseudomonas aeruginosa | 169,5000,000 | <10 | >99.99% |
| (ATCC 15442) | Log 8.23 | <Log 1.00 | (>7.23) |
| Staphylococcus aureus | 212,500,000 | <10 | >99.99% |
| (ATCC 6538) | Log 8.33 | <Log 1.00 | (>7.33) |
| Enterococcus gaecium | 156,500,000 | <10 | >99.99% |
| (ATCC 6057) | Log 8.20 | <Log 1.00 | (>7.19) |

### Example 4: Testing Antifungal Activity Against A. niger

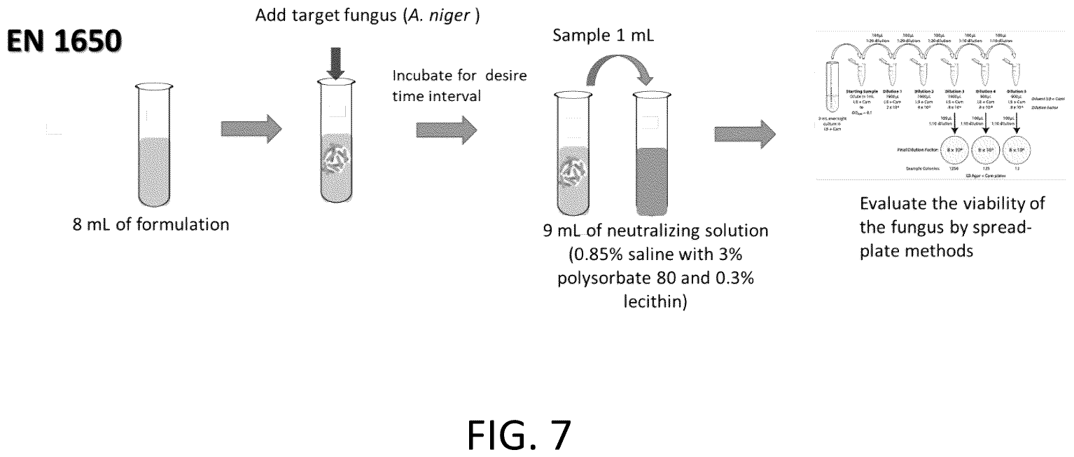

The antifungal activity of the formulation was evaluated according to the standard test EN 1650 (FIG. 7). In brief, 2 mL of Aspergillus niger (A. niger) was added into 8 mL of formulation. 1 mL of the formulation was sampled immediately and added into 9 mL of neutralizing solution. After 15 min, another 1 mL of the formulation was sampled again and added into 9 mL of neutralizing solution. The neutralizing solution was made up of 0.85% saline with 3% polysorbate 80 and 0.3% lecithin. Finally, the viability A. niger of the neutralized sample was determined with the spread plate method using potato dextrose agar as culture medium. The antifungal activity of the formulations was listed in Table 4.

TABLE 4
(Antifungal activity of different
formulation against A. niger):

| Formulation | Antifungal activity (%) |
|---|---|
| AF37 | 99.8 ± 0.3 |
| AF38 | 99.9 ± 0.1 |
| AF39 | 99.7 ± 0.4 |
| AF40 | 97.4 ± 2.1 |
| AF41 | 97.9 ± 2.0 |
| AF42 | 98.7 ± 1.0 |

### Example 5: Testing Antifungal Activity Against C. albicans and A. Brasiliensis

The antifungal effect of AF42 was tested in accordance with international standard EN 1650: 2019 Chemical disinfectants and antiseptics—Quantitative suspension test for the evaluation of fungicidal or yeasticidal activity of chemical disinfectants and antiseptics used in food, industrial, domestic and institutional areas.

The results are shown in Table 5 as below.

TABLE 5
(Test results of antibacterial activity of AF42 against
C. albicans and A. brasiliensis):

| | | Counts of test | |
|---|---|---|---|
| | Counts | sample at | Percentage |
| | of test | concentration | reduction |
| Test microorganism | suspension | 80% | (log reduction) |
| Candida albicans | 40,000,000 | 38 | >99.99% |
| (ATCC 10231) | Log 7.60 | Log 1.58 | (6.02) |
| Aspergillus brasiliensis | 40,000,000 | 24,500 | 99.94% |
| (ATCC 16404) | Log 7.60 | <Log 3.21 | (3.21) |

Remarks to the tests:
Test temperature: Ambient
Test contact time: 60 min
Test material concentration: 80%
Diluent used for product test solution: Distilled water
Neutralizer: 0.85% saline with 3% polysorbate 80 and 0.3% lecithin
Type of media used: Malt Extract Agar
Incubation condition: 30±1° C. for 48-72 h
Test Method: Dilution-Neutralization method
Test validation:
Experimental condition control (A) Valid
Neutralizer control (B) Valid
Method validation (C) Valid
Interfering substance: None

### Example 6: Aerosol Size Determination

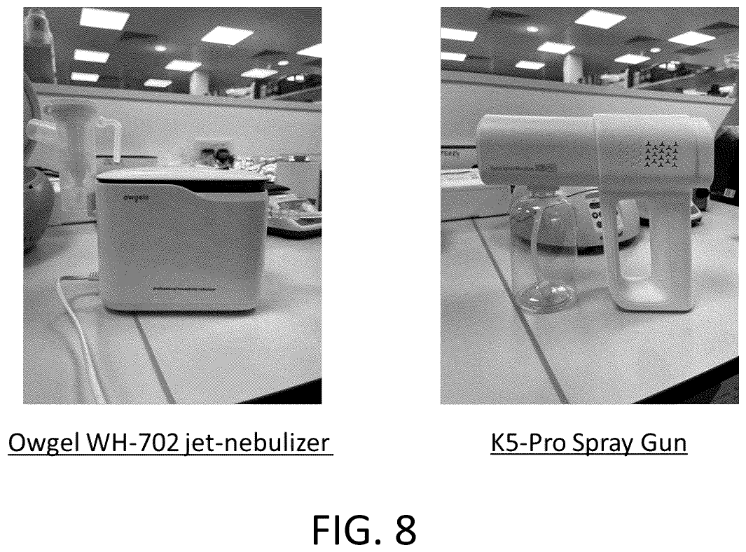

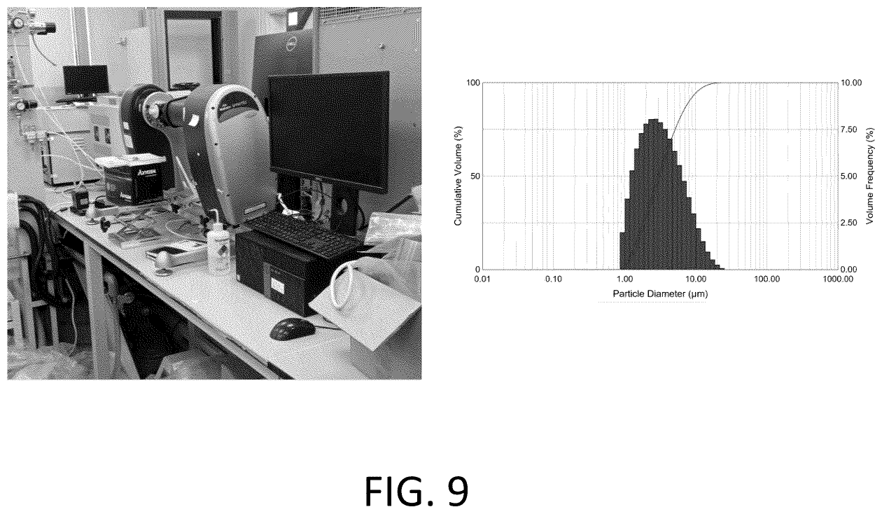

The formulations were vaporized by an Owgel WH-702 jet-nebulizer, K5-Pro Spray Gun or Muji ultrasonic nebulizer (FIG. 8). The aerosol size of the vaporized formulation was then determined by a Spraytec laser diffraction system (Malvern Panalytical) (FIG. 9). Spraytec laser diffraction system allows measurement of spray particle and spray droplet size distributions in real-time using the principle of laser diffraction. The measured aerosol size of formulations was listed in Table 6, Table 7 and Table 8.

TABLE 6
(Dv10, Dv50 and Dv90 of different formulation
using Owgel WH-702 jet-nebulizer):

| | | AF37 | AF38 | AF39 | AF40 | AF41 | AF42 | AF43 | ASIS002 |
|---|---|---|---|---|---|---|---|---|---|
| Particle | Dv10 | 1.322 | 1.227 | 1.2 | 1.151 | 1.516 | 1.553 | 1.665 | 2.305 |
| Diameter | Dv50 | 3.044 | 2.409 | 2.223 | 2.032 | 3.434 | 3.629 | 4.033 | 5.985 |
| (μm) | Dv90 | 8.038 | 6.125 | 5.188 | 4.694 | 8.52 | 9.975 | 9.811 | 14.05 |

TABLE 7
(Dv10, Dv50 and Dv90 of different formulation
using K5-Pro Spray Gun):

| | | AF37 | AF43 |
|---|---|---|---|
| Particle | Dv10 | 9.476 | 10.31 |
| Diameter | Dv50 | 32.6 | 37.57 |
| (μm) | Dv90 | 73.33 | 77.19 |

TABLE 8
(Dv10, Dv50 and Dv90 of different formulation
using Muji ultrasonic nebulizer):

| | | Sanimood 001 |
|---|---|---|
| Particle | Dv10 | 2.061 |
| Diameter | Dv50 | 4.259 |
| (μm) | Dv90 | 12.19 |

### Example 7: Testing Surface Bacterial Inactivation Against S. aureus

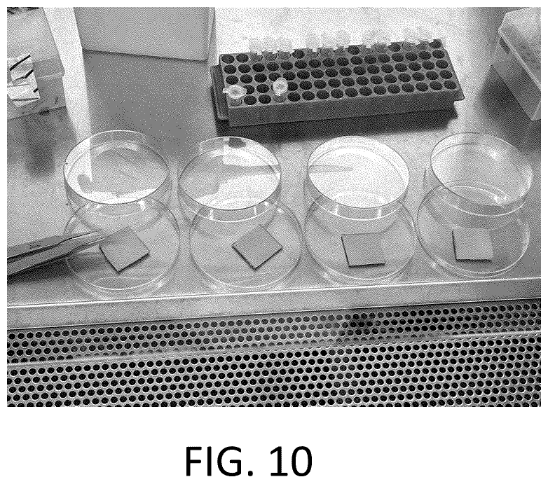

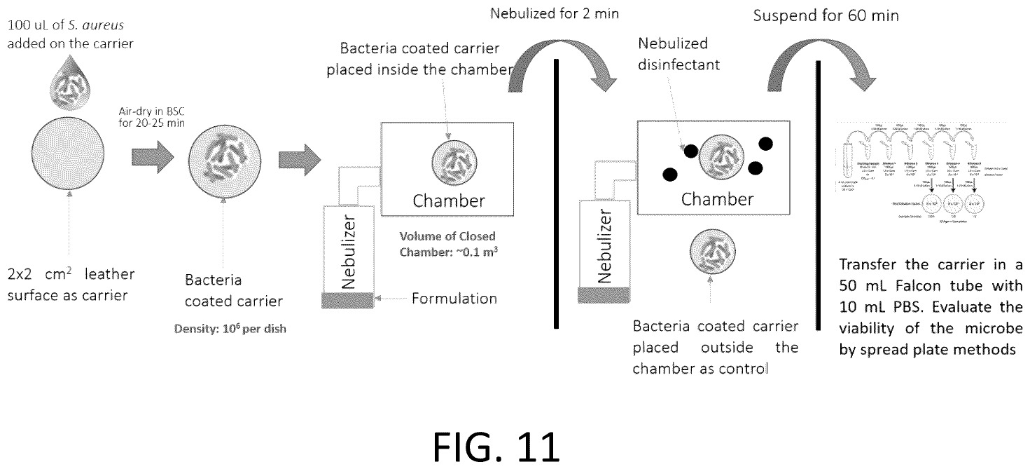

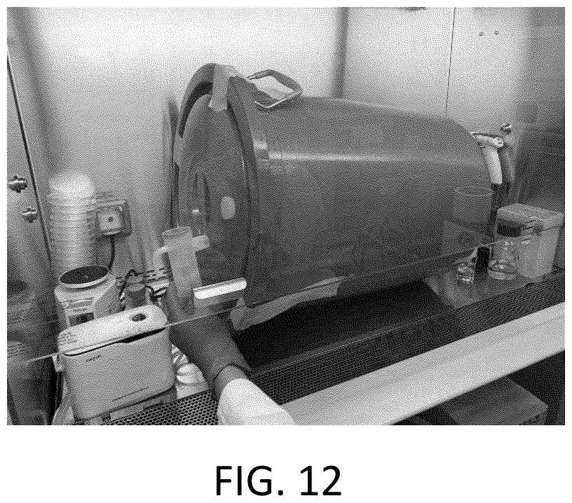

The surface inactivation efficiency of vaporized formulation was determined with procedures modified from EN 17272. In brief, 100 μL of 10⁷ CFU/mL of S. aureus was spread on carrier. A piece of 2×2 cm² leather was used as the carrier (FIG. 10). The carrier was left for 20-25 min in a biological safety cabinet to evaporate the water on the carrier surface. Two carriers coated with S. aureus were first placed outside the chamber to serve as a control to eliminate the impact of bacterial inactivation resulting from drying. Then two S. aureus coated carriers were placed into a closed chamber (volume: 87 dm³). The formulation was vaporized by an Owgel WH-702 jet-nebulizer at a rate of 0.6 mL/min and the nebulization time was 2 min. After the nebulization process, the chamber was left for 60 min to allow the vaporized formulation to disinfect the carrier. After 60 min, the carriers were transferred into a 50 mL Falcon tube with 10 mL phosphate-buffered saline (PBS) inside, respectively. The Falcon tubes were vortexed for 30 s to wash the S. aureus on the carriers into the PBS. Finally, the viability of the S. aureus in PBS was determined by spread plate method using tryptic soy agar as culture medium. The procedures on the evaluation of surface inactivation efficiency were elaborated in FIG. 11. The in-house surface inactivation efficiency testing setup was shown in FIG. 12.

The surface inactivation efficiency of the formulation against S. aureus is determined by compare the colony forming unit (CFU) between the control and treatment as below:

$$Surface inactivation efficiency = ( CFU in control - CFU in treatment ) \times 100 \% \qquad ( Equation 1 )$$

TABLE 9
(Surface inactivation activity different
formulation against S. aureus):

| Formulation | Surface bacterial inactivation efficiency (%) |
|---|---|
| AF37 | 75.8 ± 6.8 |
| AF38 | 83.1 ± 22.0 |
| AF39 | 71.4 ± 14.0 |
| AF40 | 84.8 ± 3.6 |
| AF41 | 95.5 ± 3.3 |
| AF42 | 98.6 ± 1.1 |

### Example 8: Testing Surface Fungal Inactivation Against A. niger

The surface fungal inactivation efficiency of vaporized formulation was determined with procedures modified from EN 17272. The procedures were similar to that listed in example 5. In brief, 100 μL of 10⁷ spore/mL of A. nigers was spread on carrier. A piece of 2×2 cm² leather was used as the carrier. The carriers would be first left for 20-25 min in a biological safety cabinet to evaporate the water on the carrier surface. Two carriers coated with A. niger were placed outside the chamber to serve as a control to eliminate the impact of fungal inactivation attributed to drying. Then, two A. niger coated carriers were placed into a closed chamber (volume 87 dm³). The formulation was vaporized by a K5-Pro spray gun at a rate of 10 mL/min and the nebulization time was 12 or 20 s. After the nebulization process, the chamber was left for 4 h to allow the vaporized formulation to disinfect the carrier. After 4 h, the carriers were transferred into a 50 mL Falcon tube with 10 mL PBS inside, respectively. The Falcon tubes were vortexed for 30 s to wash the A. niger on the carriers into the PBS. Finally, the viability of the A. niger in PBS was determined by spread plate method using potato dextrose agar as culture medium.

The surface inactivation against A. niger was calculated with Equation 1 shown in Example 5.

TABLE 10
(Surface inactivation activity different
formulation against A. niger):

| | Surface fungal inactivation | |
|---|---|---|
| Formulation | efficiency (%) | Nebulization time (s) |
| AF37 | 71.5 ± 9.5 | 12 |
| AF38 | 63.5 ± 9.5 | 12 |
| AF39 | 61.9 ± 4.4 | 12 |
| AF40 | 72.3 ± 1.5 | 12 |
| AF41 | 73.8 ± 3.1 | 12 |
| AF42 | 80.5 ± 3.4 | 12 |
| AF42 | 96.7 ± 2.9 | 20 |
| AF43 | 96.9 ± 1.8 | 20 |

### Example 9: Testing Airborne Bacterial Inactivation

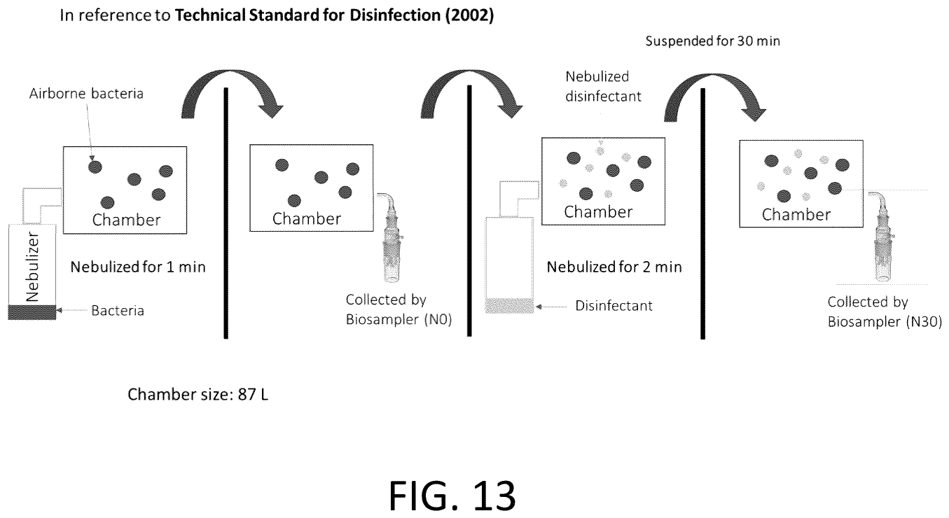

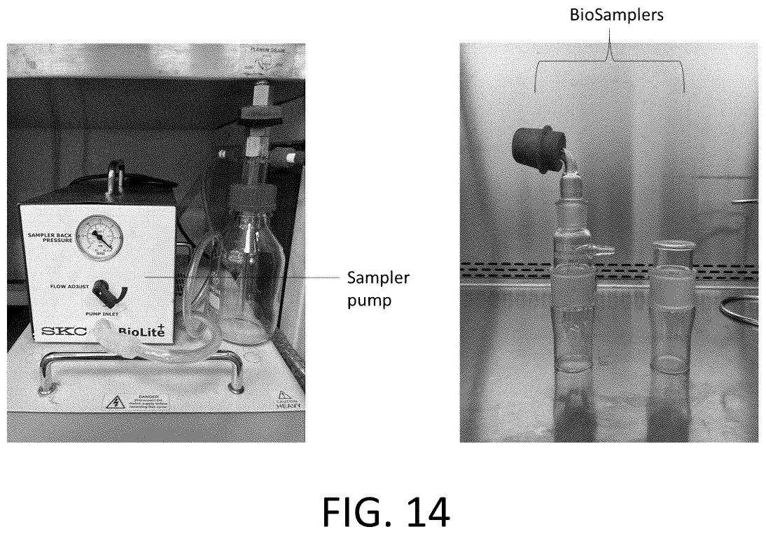

The airborne bacterial inactivation efficiency of vaporized formulation was determined with procedures modified from Technical Standard for Disinfection (2002) Section 2.2.3. (FIG. 13). In brief, a closed chamber (volume 87 dm³) was placed in a biological safety cabinet. Airborne Staphylococcus epidermidis was sprayed by nebulizing a S. epidermidis solution using an Owgel WH-702 jet-nebulizer at a rate of 0.6 mL/min for 1 min. After the injection of airborne S. epidermidis into the chamber, air sample was collected by a BioSampler with 20 mL PBS at a rate of 12.5 L/min for 30 s (FIG. 14). Then the sanitizer was vaporized by Owgel WH-702 jet-nebulizer at a rate of 0.6 mL/min for 2 min. The chamber was left for 30 min for the inactivation process. After 30 min, air sample was collected again by a BioSampler with 20 mL PBS at a rate of 12.5 L/min for 30 s.

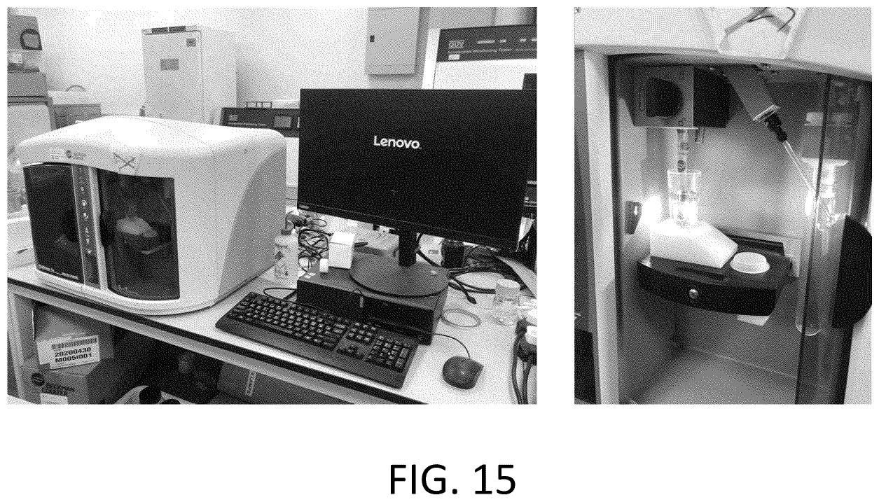

After each collection of air sample, 1 mL of PBS inside the BioSampler was immediately transferred to 9 mL of neutralizing solution. The neutralizing solution was made up of 0.85% saline with 3% polysorbate 80 and 0.3% lecithin. Then the viable count of S. epidermidis in PBS was determined by spread plate method using tryptic soy agar as culture medium. To normalize the number of the bacterial count collected from each trial, the total count of S. epidermidis in PBS was determined by a Multisizer 4e Coulter Counter (FIG. 15). The survival ratio of the S. epidermidis was calculated according to Equation 2. The airborne bacterial inactivation efficiency was determined by comparing the survival ratio before and after the sanitization process (Equation 3).

$$Survival ratio ( N ) = Viable count / Total count \qquad Equation 2$$

$$Airborne bacterial inactivation efficiency = ( {N}_{0 min} - {N}_{30 min} ) / {N}_{0 min} \times 100 \% \qquad Equation 3$$

The airborne bacterial inactivation efficiency against S. epidermidis was shown in Table 11.

TABLE 11
(Airborne bacterial inactivation efficiency of the formulation):

| Formulation | Airborne bacterial inactivation efficiency (%) |
|---|---|
| AF42 | 99.5 ± 1.0 |
| ASIS 002 | 99.0 ± 0.6 |

### Example 10: Testing for Dermal Toxicity

The dermal toxicity of AF42 was tested with reference to the test standard of OECD-402: Acute dermal toxicity (adopted 9 Oct. 2017).

The results are shown in Tables 12 and 13 below.

TABLE 12
(Body weight changes and toxic signs for animals after dosing):

| Dose | | | Body weight (g) | | | Toxic signs | Toxic | Death | Gross |
|---|---|---|---|---|---|---|---|---|---|
| (mg/kg · bw) | Sex | No. | 0 d | 7 d | 14 d | occur time | Signs | time | necropsy |
| 2000 | female | 1 | 237.8 | 260.1 | 280.5 | — | N | — | — |
| | | 2 | 229.3 | 254.5 | 265.0 | — | N | — | — |
| | | 3 | 229.2 | 247.9 | 266.7 | — | N | — | — |

| Body weight | Mean | 232.1 | 254.2 | 270.7 | — | — | — |
|---|---|---|---|---|---|---|---|
| changes | SD | 4.9 | 6.1 | 8.5 | — | — | |

(“N” means no obvious abnormality)

TABLE 13
(Results of acute dermal toxicity test in rats):

| Dose (mg/kg · bw) | Test animals | Death animals | Mortality rate |
|---|---|---|---|
| 2000 | 0 | 0 | 0.0 |

### Example 11: Testing for Dermal Irritation/Corrosion

The dermal irritation/corrosion effects of AF42 was tested with reference to standard test OECD-404: Acute dermal irritation (adopted 2015).

Test results are shown in Table 14 as below.

TABLE 14
(The scores of the test substance):

| | | | 1 h | | | | 24 h | | | |
|---|---|---|---|---|---|---|---|---|---|---|
| Animal | | Weight | Test substance | | Control | | Test substance | | Control | |
| no. | Sex | (kg) | Eythemaeschar | Edema | Eythemaeschar | Edema | Eythemaeschar | Edema | Eythemaeschar | Edema |
| 1 | female | 2.2 | 1 | 0 | 0 | 0 | 0 | 0 | 0 | 0 |
| 2 | female | 2.4 | 0 | 0 | 0 | 0 | 0 | 0 | 0 | 0 |
| 3 | female | 2.3 | 0 | 0 | 0 | 0 | 0 | 0 | 0 | 0 |
| | | | 48 h | | | | 72 h | | | |
| Animal | | Weight | Test substance | | Control | | Test substance | | Control | |
| no. | Sex | (kg) | Eythemaeschar | Edema | Eythemaeschar | Edema | Eythemaeschar | Edema | Eythemaeschar | Edema |
| 1 | female | 2.2 | 1 | 0 | 0 | 0 | 0 | 0 | 0 | 0 |
| 2 | female | 2.4 | 0 | 0 | 0 | 0 | 0 | 0 | 0 | 0 |
| 3 | female | 2.3 | 0 | 0 | 0 | 0 | 0 | 0 | 0 | 0 |

Remarks:
Erythema and Eschar Formation:
0: No Erythema
1: Very slight erythema (barely perceptible)
2: Well defined erythema
3: Moderate to severe erythema
4: Severe erythema (beef redness) to eschar formation preventing grading of erythema
Oedema Formation:
0: No oedema
1: Very slight oedema (barely perceptible)
2: Slight oedema (edges of area well defined by definite raising)
3: Moderate oedema (raised approximately 1 mm)
4: Severe oedema (raised more than 1 mm and extending beyoud area of exposure)

### Example 12: Testing Acute Inhalation Toxicity

The dermal irritation/corrosion of AF42 was tested with reference to standard test OECD-403: Acute inhalation toxicity (adopted: 7 Sep. 2009).

The test results are shown in Table 15 as below.

TABLE 15
(Body weight chagnes response data and dose level for animals after exposure):

| | Concentrations | Test | Body Weight (g) | | | | | Death | Mortality |
|---|---|---|---|---|---|---|---|---|---|
| Sex | (mg/m³) | animals (n) | 0 d | 1 d | 3 d | 7 d | 14 d | animals (n) | (%) |
| Female | 5000 | 3 | 20.7 ± 0.2 | 20.8 ± 0.3 | 23.2 ± 0.6 | 26.9 ± 1.2 | 32.6 ± 1.0 | 0 | 0 |
| Male | 5000 | 3 | 20.5 ± 0.3 | 21.2 ± 0.3 | 24.8 ± 0.5 | 29.9 ± 1.6 | 39.5 ± 1.2 | 0 | 0 |

### Example 13: Testing for Substances of Very High Concern (SVHC)

The contents of AF42 were analyzed by accredited laboratory by using methods including ICP-OES, UV-VIS, GC-MS, HPLC-DAD/MS and colorimetric method.

The test results are shown in Table 16 as below.

TABLE 16
(SVHC content of AF42):

| Test item(s) | Test result(s)% | RL(%) |
|---|---|---|
| All tested SVHC in candidate list | ND | — |
| All tested Potential SVHC | ND | — |

### Example 14: Testing for Harmful Volatile Organic Compound (VOC) Emission

The potential of emission of harmful VOC of AF42 was analyzed according to standard test USP 467 Residual solvents.

The test results are shown in Table 17 below.

TABLE 17
(Results of harmful VOC content in AF42):

| Test item | Result (mg/kg) | MDL (mg/kg) |
|---|---|---|
| Benzene | ND | 2 |
| Carbon tetrachloride | ND | 4 |
| 1,2-Dichloroethane | ND | 5 |
| 1,1-Dichloroethene | ND | 8 |
| 1,1,1-Trichloroethane | ND | 1500 |
| Acetonitrile | ND | 410 |
| Chlorobenzene | ND | 360 |
| Chloroform | ND | 60 |
| Cyclohexane | ND | 3880 |
| 1,2-Dichloroethene | ND | 1870 |
| 1,2-Dimethoxyethane | ND | 100 |
| N,N-Dimethylacetamide | ND | 1090 |
| N,N-Dimethylformamide | ND | 880 |
| 1,4-Dioxane | ND | 380 |
| 2-Ethoxyethanol | ND | 160 |
| Ethylene glycol | ND | 620 |
| Formamide | ND | 220 |
| Hexane | ND | 290 |
| Methanol | ND | 3000 |
| 2-Methoxyethanol | ND | 50 |
| Methylbutylketone | ND | 50 |
| Methylcyclohexane | ND | 1180 |
| Methylene chloride | ND | 600 |
| N-Methylpyrrolidone | ND | 530 |
| Nitromethane | ND | 50 |
| Pyridine | ND | 200 |
| Sulfolane | ND | 160 |
| Tetrahydrofuran | ND | 720 |
| Tetralin | ND | 100 |
| Toluene | ND | 890 |
| Trichloroethylene | ND | 80 |

(Remarks: MDL = Method detection limit; ND = Not detected (<MDL))

### Example 15: Testing for Restriction of Hazardous Substances (RoHS)

The content of AF42 was checked with reference to standard tests IEC 62321-4:2013+A1:2017, IEC 62321-5:2013, IEC 62321-7-2:2017, IEC 62321-6:2015 and IEC 62321-8_2017, analyzed by ICP-OES, UV-Vis and GC-MS.

The test results are shown in Table 18 as below.

TABLE 18
(RoHS content of AF42):

| | Limit | result(s) | MDL |
|---|---|---|---|
| Test item(s) | (mg/kg) | Test | (mg/kg) |
| Cadmium (Cd) | 100 | ND | 2 |
| Lead (Pb) | 1000 | ND | 2 |
| Mercury (Hg) | 1000 | ND | 2 |
| Hexavalent Chromium (CrVI) | 1000 | ND | 8 |
| Sum of PBBs | 1000 | ND | — |
| Monobromobiphenyl | / | ND | 5 |
| Dibromobiphenyl | / | ND | 5 |
| Tribromobiphenyl | / | ND | 5 |
| Tetrabromobiphenyl | / | ND | 5 |
| Pentabromobiphenyl | / | ND | 5 |
| Hexabromobiphenyl | / | ND | 5 |
| Heptabromobiphenyl | / | ND | 5 |
| Octabromobiphenyl | / | ND | 5 |
| Nonabromobiphenyl | / | ND | 5 |
| Decabromobiphenyl | / | ND | 5 |
| Sum of PBDEs | 1000 | ND | — |
| Monobromodiphenyl ether | / | ND | 5 |
| Dibromodiphenyl ether | / | ND | 5 |
| Tribromodiphenyl ether | / | ND | 5 |
| Tetrabromodiphenyl ether | / | ND | 5 |
| Pentabromodiphenyl ether | / | ND | 5 |
| Hexabromodiphenyl ether | / | ND | 5 |
| Heptabromodiphenyl ether | / | ND | 5 |
| Octabromodiphenyl ether | / | ND | 5 |
| Nonabromodiphenyl ether | / | ND | 5 |
| Decabromodiphenyl ether | / | ND | 5 |
| Dibutyl Phthalate (DBP) | 1000 | ND | 50 |
| Butyl benzyl phthalate (BBP) | 1000 | ND | 50 |
| Bis-(2-ethylhexyl) Phthalate (DEHP) | 1000 | ND | 50 |
| Diisobutyl Phthalates (DIBP) | 1000 | ND | 50 |

(Remarks: 1 mg/kg = 0.0001%; MDL = Method Detection Limit; ND = Not Detected (<MDL))

### Example 18: Testing for Metal Corrosion

The corrosion rate of AF42 on different metal was evaluated according to Technical Standard for Disinfection (2002) Section 2.2.4.

The test results are shown in Table 19 as below, with Table 20 as remarks.

TABLE 19
(Corrosion rate of AF42 on different metals):

| | Corrosion | Method detection | |
|---|---|---|---|
| Test item (s) | rate (mm/a) | limit (mm/a) | Comment |
| Carbon steel | 0.08 | 0.01 | Slight corrosion |
| Copper | 0.03 | 0.01 | Slight corrosion |
| Aluminum | 0.04 | 0.01 | Slight corrosion |
| Stainless steel | <0.01 | 0.01 | Basically no |

TABLE 20
(Corrosion classification standard in Technical
Standard for Disinfection (2002) Section 2.2.4):

| Corrosion Rate (mm/a) | Level |
|---|---|
| <0.01 | Basically no |
| 0.01-<0.1 | Slight corrosion |
| 0.1-<1.0 | Moderate corrosion |
| ≥1.0 | Severe corrosion |

### Example 17: Testing for Airborne Bacterial Disinfection Efficiency

The airborne disinfection efficiency of ASIS002 against bacteria was evaluated according to Technical Standard for Disinfection (2002) Section 2.1.3.4. The evaluation was used into two volume of chambers, 1 m³ and 20 m³.

The test results are shown in Tables 21 and 22 as below.

TABLE 21
(Airborne disinfection efficiency of ASIS002 in volume of 1 m³):

| | | Control group | | Test group | |
|---|---|---|---|---|---|
| | | Number | | Number | |
| | | of viable | Natural | of viable | |
| Action | Test | colonies | extinction | colonies | Killing |
| time | group | (cfu/m³) | rate (%) | (cfu/m³) | rate (%) |
| 0 h | 1 | 3.2 × 10⁶ | / | 3.6 × 10⁶ | / |
| | 2 | 3.4 × 10⁶ | / | 3.5 × 10⁶ | / |
| | 3 | 3.5 × 10⁶ | / | 3.3 × 10⁶ | / |
| 2 h | 1 | 1.5 × 10⁶ | 53.12 | 4.0 × 10⁴ | 97.63 |
| | 2 | 1.6 × 10⁶ | 52.94 | 3.8 × 10⁴ | 97.69 |
| | 3 | 1.5 × 10⁶ | 57.14 | 3.6 × 10⁴ | 97.45 |

Remarks:
Volume of the aerosol chamber: 1 m³
Test strain: Staphylococcus albus 8032, 3^{rd} generation
Medium: Nutrient agar
Components of neutralizer: PBS solution containing 0.283% anhydrous disodium hydrogen phosphate, 0.136% potassium dihydrogen phosphate, 5% Tween-80, 3% lecithin, 1% saponyl, 1% glycine
Test conditions: temperature 22.4° C., relative humidity 60%)

TABLE 22
(Airborne disinfection efficiency of ASIS02 in volume of 20 m³):

| | | Control group | | Test group | |
|---|---|---|---|---|---|
| | | Number | | Number | |
| | | of viable | Natural | of viable | |
| Action | Test | colonies | extinction | colonies | Killing |
| time | group | (cfu/m³) | rate (%) | (cfu/m³) | rate (%) |
| 0 h | 1 | 1.3 × 10⁵ | / | 1.4 × 10⁵ | / |
| | 2 | 1.2 × 10⁵ | / | 1.5 × 10⁵ | / |
| | 3 | 1.3 × 10⁵ | / | 1.5 × 10⁵ | / |
| 2 h | 1 | 5.7 × 10⁴ | 56.15 | 1.9 × 10³ | 96.91 |
| | 2 | 5.0 × 10⁴ | 58.33 | 1.7 × 10³ | 97.28 |
| | 3 | 5.3 × 10⁴ | 59.23 | 1.3 × 10³ | 97.87 |

Remarks:
Volume of the aerosol chamber: 20 m³
Test strain: Staphylococcus albus 8032, 3^{rd} generation
Medium: Nutrient agar
Components of neutralizer: PBS solution containing 0.283% anhydrous disodium hydrogen phosphate, 0.136% potassium dihydrogen phosphate, 5% Tween-80, 3% lecithin, 1% saponyl, 1% glycine
Test conditions: temperature 23.0° C., relative humidity 63%

### Example 18: Testing Antiviral Activity

The antiviral effect of ASIS002 was evaluated in accordance with standard Technical Standard for Disinfection (2002) Section 2.1.1.1.

The test results are shown in Table 23 as below.

TABLE 23
(Antiviral activity of ASIS002 against Influenza A virus H1N1):

| | Average logarithm | | |
|---|---|---|---|
| | of infectivity titre | Logarithm | Virus |
| | value control group | reduction | inactivation |
| Run | (lgTCID₅₀/mL) | value | ratio (%) |
| 1 | 5.84 | >4.00 | >99.99 |
| 2 | 5.82 | >4.00 | >99.99 |
| 3 | 5.85 | >4.00 | >99.99 |
| Average | 5.84 | >4.00 | >99.99 |

Remarks to the test:
The cells in negative control groups grew well
Test strain: Influenza A virus H1N1:A/PR/8/34 (ATCC VR-1469)
Host cell: MDCK cells
Action time: 2 hours)

### Example 19: Testing for Airborne Viral Disinfection Efficiency

The airborne disinfection efficiency of Sanimood 001 against virus was evaluated according to Technical Standard for Disinfection (2002) Section 2.1.3.4. The evaluation was used into chambers of 10 m³.

The test results are shown in Tables 24 as below.

TABLE 24
(Airborne disinfection efficiency
of Sanimood001 in volume of 10 m³):

| | | Control group | | Test group | |
|---|---|---|---|---|---|
| | | Air Virus | Natural | Air Virus | |
| Action | Test | content | extinction | content | Killing |
| time | group | (TCID₅₀/m³) | rate (%) | (TCID₅₀/m³) | rate (%) |
| 0 h | 1 | 2.33 × 10⁶ | / | 2.44 × 10⁶ | / |
| | 2 | 3.08 × 10⁶ | / | 1.99 × 10⁶ | / |
| | 3 | 2.44 × 10⁶ | / | 2.08 × 10⁶ | / |
| 2 h | 1 | 9.73 × 10⁵ | 58.31 | 9.73 × 10² | 99.90 |
| | 2 | 1.20 × 10⁶ | 61.10 | 7.91 × 10² | 99.90 |
| | 3 | 8.28 × 10⁵ | 66.12 | 6.00 × 10² | 99.91 |

Remarks:
Volume of the aerosol chamber: 10 m³
Dosage: 10 mL/m³
Test strain: Influenza A virus H1N1:A/PR/8/34 (ATCC VR-1469) Host cell: MDCK
Components of neutralizer: PBS solution containing 0.283% anhydrous disodium hydrogen phosphate, 0.136% potassium dihydrogen phosphate, 5% Tween-80, 3% lecithin, 1% saponyl, 1% glycine

### Example 20: Evaluating Antibacterial and Antifungal Activity in Vehicle Cabin

The surface and airborne microbial disinfection activity of AF43 was evaluated in vehicle cabin. Twelve taxis with size of about 2 m³ were chosen for the test. In the vehicle cabin, ability of AF43 to disinfect airborne and surface microbes was assessed. For the test, 12 taxis with a volume of roughly 2 m³ were selected. First, 100 L of air was pumped through the tryptic soy agar and potato dextrose agar, respectively, to collect samples of the total amount of airborne bacteria and fungi. Then, the microbes on the surfaces in four locations with an area of 20×20 cm² were sampled using swabs. A leather surface with artificially inoculated A. niger was placed on the driver seat (all sampling locations were shown in FIG. 5). The air conditioner of testing vehicle was then turned on, and the AF43 was nebulized for 5 min using a K5-Pro spray gun. The spray gun was pointed at the front right outlet of air-conditioner and the nebulized formulation was circulated by the airflow generated by the air-conditioner. After the formulation has been nebulized, the air conditioner was turned on for a further 3 min to circulate the vaporized formulation. After that, the air conditioner was switched off, and the car was allowed to sit for 2 h. After 2 h incubation, the surface and airborne microbe was sampled again with aforementioned methods. The collected bacteria and fungi were evaluated using spread plate methods on a tryptic soy agar and potato dextrose agar, respectively. All agars were incubated at 37° C. for 48 h for colony formation. The surface and airborne microbial disinfection efficiency was evaluated by comparing the difference between the colony formation unit of the surface and airborne microbes before and after the nebulization of disinfectants.

The test results are shown in Table 25 and 26 below.

TABLE 25
Summary table for the reduction of viable fungi

| | Reduction of viable fungi (%) | | | | | | | | | | | | Total |
|---|---|---|---|---|---|---|---|---|---|---|---|---|---|
| | Taxi | Taxi | Taxi | Taxi | Taxi | Taxi | Taxi | Taxi | Taxi | Taxi | Taxi | Taxi | aver- |
| Locations | 1 | 2 | 3 | 4 | 5 | 6 | 7 | 8 | 9 | 10 | 11 | 12 | age |
| Front left | — | 100 | — | 100 | 99 | 90 | — | — | — | 0 | 100 | — | |
| outlet | | | | | | | | | | | | | |
| Front | 93 | 97 | — | — | 62 | — | — | 100 | — | — | — | — | |
| middle | | | | | | | | | | | | | |
| outlet | | | | | | | | | | | | | |
| Front right | 100 | — | 92 | — | — | — | — | — | — | — | — | — | |
| outlet | | | | | | | | | | | | | |
| Boot lid | 82 | 97 | 96 | — | 70 | — | — | 58 | 62 | 45 | 100 | 95 | |
| Rear Seat | 100 | — | 95 | 100 | 42 | 0 | 99 | 55 | 96 | — | — | 93 | |
| Leather | 52 | 44 | 88 | 96 | 47 | 50 | 70 | 68 | 44 | 50 | 62 | 62 | |
| Average | 85 | 85 | 93 | 99 | 64 | 47 | 84 | 70 | 67 | 32 | 87 | 83 | 75 |
| surface | | | | | | | | | | | | | |
| disinfection | | | | | | | | | | | | | |
| efficacy | | | | | | | | | | | | | |
| Air sample | 0 | 0 | 0 | 47 | 89 | 46 | — | 71 | — | 100 | 85 | 85 | 52 |

Remarks:
1. Fungal samples with CFU > 10 either before or after treatment were selected for calculation of antimicrobial efficacy. Cells in the summary table for the samples that do not fulfill selection criteria were left blank.
2. Negative reduction was labelled as zero.

TABLE 26
Summary table for the reduction of viable bacteria

| | Reduction of viable bacteria (%) | | | | | | | | | | | | Total |
|---|---|---|---|---|---|---|---|---|---|---|---|---|---|
| | Taxi | Taxi | Taxi | Taxi | Taxi | Taxi | Taxi | Taxi | Taxi | Taxi | Taxi | Taxi | aver- |
| Locations | 1 | 2 | 3 | 4 | 5 | 6 | 7 | 8 | 9 | 10 | 11 | 12 | age |
| Front left | 100 | — | — | 91 | — | — | — | — | — | — | — | — | |
| outlet | | | | | | | | | | | | | |
| Front | — | 100 | 100 | 100 | — | — | — | — | — | — | — | — | |
| middle | | | | | | | | | | | | | |
| outlet | | | | | | | | | | | | | |
| Front right | — | — | — | 100 | — | — | — | — | — | — | — | — | |
| outlet | | | | | | | | | | | | | |
| Boot lid | 100 | 87 | 89 | 100 | 96 | 90 | 96 | — | — | 81 | — | 61 | |
| Rear Seat | — | 90 | 93 | 0 | 99 | 56 | 100 | 69 | 82 | — | — | 100 | |
| Average | 100 | 92 | 94 | 78 | 97 | 73 | 98 | 69 | 82 | 81 | — | 81 | 86 |
| surface | | | | | | | | | | | | | |
| disinfection | | | | | | | | | | | | | |
| efficacy | | | | | | | | | | | | | |
| Air sample | — | — | — | 92 | — | — | — | — | — | — | — | — | 92 |

Remarks:
1. Bacteria samples with CFU > 20 either before or after treatment were selected for calculation of antimicrobial efficacy. Cells in the summary table for the samples that do not fulfill selection criteria were left blank.
2. Negative reduction was labelled as zero.

In summary, the reduction of surface viable fungi ranged from 32% to 99% and the reduction of airborne viable fungi ranged from 0% to 100%. The average reduction of surface and airborne fungi was 75% and 52%, respectively. The reduction of surface viable bacteria ranged from 69% to 100% and the reduction of airborne viable bacteria was 92%. The average reduction of surface and airborne bacteria was 86% and 92%, respectively. The results showed that AF43 developed formulation can significantly reduce the viable microorganisms in vehicle cabins.

It should be understood that the examples and embodiments described herein are for illustrative purposes only and that various modifications or changes in light thereof will be suggested to persons skilled in the art and are to be included within the spirit and purview of this application.

All patents, patent applications, provisional applications, and publications referred to or cited herein are incorporated by reference in their entirety, including all figures and tables, to the extent they are not inconsistent with the explicit teachings of this specification.

It should be understood that numerous specific details, relationships, and methods are set forth to provide a full understanding of the invention. One having ordinary skill in the relevant art, however, will readily recognize that the invention can be practiced without one or more of the specific details or practiced with other methods, protocols, reagents, cell lines and animals. The present invention is not limited by the illustrated ordering of acts or events, as some acts may occur in different orders and/or concurrently with other acts or events. Furthermore, not all illustrated acts, steps or events are required to implement a methodology in accordance with the present invention. Many of the techniques and procedures described, or referenced herein, are well understood and commonly employed using conventional methodology by those skilled in the art.

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly used dictionaries, should be interpreted as having a meaning that is consistent with their meaning in the context of the relevant art and/or as otherwise defined herein.

1. A surface-aerosol interface sanitization system for automated sanitization of interior environments, comprising:
a nebulizer selected from a jet-nebulizer or ultrasonic nebulizer configured to emit droplets having a droplet size of 1 μm to 14 μm, or a spray gun configured to emit droplets having a droplet size of 9 μm to 80 μm; and
a non-toxic, non-corrosive and biocompatible sanitizer formulation comprising:
an organic acid,
a peptide with antiviral and antimicrobial properties,
a polymer binder,
a surfactant; and
an essential oil; and
wherein the sanitizer formulation is an emulsion formed by membrane emulsification through a membrane emulsifier, and the emulsified particles have a particle size of 0.05 μm to 5 μm configured to be nebulized by the jet-nebulizer, ultrasonic nebulizer or the spray gun;
wherein the sanitizer formulation has a viscosity of 15 mPas to 25 mPas such that the droplets are configured to be dispersed over an area of approximately 1 cubic meter without evaporating and having a settlement rate of 0.3 cm/s to 3 cm/s;
wherein the sanitizer formulation has at least 99% antibacterial activity against Enterococcus hirae, Escherichia coli, Pseudomonas aeruginosa, Staphylococcus aureus and Enterococcus gaecium;
wherein the sanitizer formulation has at least 95% antifungal activity against Aspergillus niger, Candida albicans and Aspergillus brasiliensis; and
wherein the sanitizer formulation has at least 99% antiviral activity against Influenza A virus H1N1.

2. The system of claim 1, wherein the organic acid in the sanitizer formulation is selected from one or more of acetic acid, citric acid, malic acid, tartaric acid, lactic acid, succinic acid or oleic acid.

3. The system of claim 1, wherein the peptide with antimicrobial and antiviral properties in the sanitizer formulation is selected from one or more of nisin, poly-L-lysine, lysozyme, lysostaphin, microcin, colicin or enterocin.

4. The system of claim 1, wherein the polymer binder in the sanitizer formulation is selected from one or more of chitosan, zein, gelatin, cellulose, pectin, alginate, acrylic latex, polyurethane or cyclodextrin.

5. The system of claim 1, wherein the surfactant in the sanitizer formulation is selected from one or more of polysorbate-based surfactants; polyglyceryl ester-based surfactants or polyglucose.

6. The system of claim 1, wherein the essential oil in the sanitizer formulation is selected from one or more of thyme oil, tea tree oil, cedar wood oil, ginger oil or lemon oil.

7. The system of claim 1, wherein the surface contact angle of the nebulized formulation droplets is less than 32%; and wherein the duration of settlement of the nebulized formulation on a surface is at least 2 hours.
